(19) United States
(12) Patent Application Publication
(10) Pub. No.: US 2021/0174509 A1
(43) Pub. Date: Jun. 10, 2021

(54) Examination Method, Examination System, and Non-Transitory Computer Readable Recording Medium

(71) Applicant: Shimadzu Corporation, Kyoto (JP)

(72) Inventors: Hiroshi IWATA, Kyoto (JP); Shigehiko ZODA, Kyoto (JP); Eiichi OZEKI, Kyoto (JP); Ippei TAKEUCHI, Kyoto (JP); Hiroomi GOTO, Kyoto (JP); Yasuko YONEDA, Kyoto (JP); Takahiro NISHIMOTO, Kyoto (JP); Toyoyuki HASHIMOTO, Kyoto (JP)

(21) Appl. No.: 17/094,254

(22) Filed: Nov. 10, 2020

(30) Foreign Application Priority Data
Dec. 6, 2019 (JP) 2019-221516

(51) Int. Cl. G06T 7/00; C12Q 1/02

(52) U.S. Cl. CPC G06T 7/0014; C12Q 1/025; G06T 2207/30072; G06T 2207/20084; G06T 2207/10056; G06T 2207/20081

(57) ABSTRACT

An object is to obtain not only information indicating whether or not a drug is effective against bacteria but also additional information about an effective drug. An examination method includes a sample preparation step of obtaining a plurality of samples by bringing drugs of a plurality of types into contact with bacteria, and an image capturing step of capturing an image of each sample. The examination method includes a step of determining efficacy of the drug based on each sample image obtained in the image capturing step, and a step of obtaining drug susceptibility information by comparing a plurality of sample images including the drug determined as being effective, in accordance with a prescribed criterion.

### BACKGROUND OF THE INVENTION

### Field of the Invention

The present disclosure relates to an examination method for examining the efficacy of a drug against bacteria, an examination system including a processor having a program for examining the efficacy installed thereon, and a non-transitory computer readable recording medium having the program recorded thereon.

### Description of the Background Art

In order to select an antibacterial drug, a susceptibility test of bacteria such as germs and fungi is conducted. “PLOS ONE”, 11 (2), Feb. 12, 2016 by Yoshimi Matsumoto, Shouichi Sakakihara, Andrey Grushnikov, Kazuma Kikuchi, Hiroyuki Noji, Akihito Yamaguchi, Ryota lino, Yasushi Yagi, and Kunihiko Nishino describes that an image of a sample obtained by bringing a drug into contact with bacteria is captured and the obtained image is processed using image analysis software, to thereby obtain the number of cells and the like from the image and determine a minimum inhibitory concentration (MIC) of the drug against the bacteria.

### SUMMARY OF THE INVENTION

However, according to the conventional method, information indicating whether or not the drug is effective against the bacteria is only obtained from the image of the sample obtained by bringing the drug into contact with the bacteria, and obtainment of other information has not been considered.

The present disclosure has been made to solve the above-described problem, and an object of the present disclosure is to obtain not only information indicating whether or not a drug is effective against bacteria but also additional information about an effective drug.

An examination method according to the present disclosure is an examination method for examining efficacy of a drug against bacteria, the examination method including: obtaining a plurality of samples, each of the plurality of samples being obtained by bringing a drug into contact with the bacteria; obtaining an image data set by capturing an image of each of the plurality of samples, the plurality of samples being different from each other in at least one condition of a drug type, a drug concentration and exposure time of the bacteria to the drug; determining the efficacy of the drug against the bacteria based on the obtained image data set; and obtaining information indicating a difference in the efficacy of the drug due to being different in the at least one condition of the drug type, the drug concentration and the exposure time of the bacteria to the drug, by extracting an image data subset from among the obtained image data set of the plurality of samples, the image data subset being for samples including the drug determined as being effective, and comparing one image with another among the image data subset in accordance with a prescribed criterion.

An examination system according to the present disclosure includes a processor having a program for examining efficacy of a drug against bacteria based on an image data set installed thereon, the image data set being obtained by capturing an image of each of a plurality of samples, each of the plurality of samples being obtained by bringing a drug into contact with the bacteria, the plurality of samples being different from each other in at least one condition of a drug type, a drug concentration and exposure time of the bacteria to the drug. The program causes the processor to perform the functions of: determining a minimum inhibitory concentration of the drug against the bacteria; obtaining information indicating a difference in the efficacy of the drug due to being different in the at least one condition of the drug type, the drug concentration and the exposure time of the bacteria to the drug; and outputting an examination result list that shows the minimum inhibitory concentration and the information indicating the difference in the efficacy.

A non-transitory computer readable recording medium according to the present disclosure has the above-described program stored thereon.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 shows a flow of examination according to the present embodiment.

FIG. 2 is a plan view of a culture plate.

FIG. 3 shows a schematic configuration of an examination device including a microscope camera according to the present embodiment.

FIG. 4 is a schematic view showing one example of a hardware configuration of an information processing device.

FIG. 5 is a block diagram showing one example of a functional configuration of the information processing device.

FIG. 6 shows an example of comparison based on a drug type.

FIG. 7 shows an example of comparison based on a drug concentration.

FIG. 8 shows an example of comparison based on the exposure time.

### DESCRIPTION OF THE PREFERRED EMBODIMENTS

An embodiment of the present disclosure will be described in detail hereinafter with reference to the drawings, in which the same or corresponding portions are denoted by the same reference characters and description thereof will not be repeated.

[Overview of Examination]

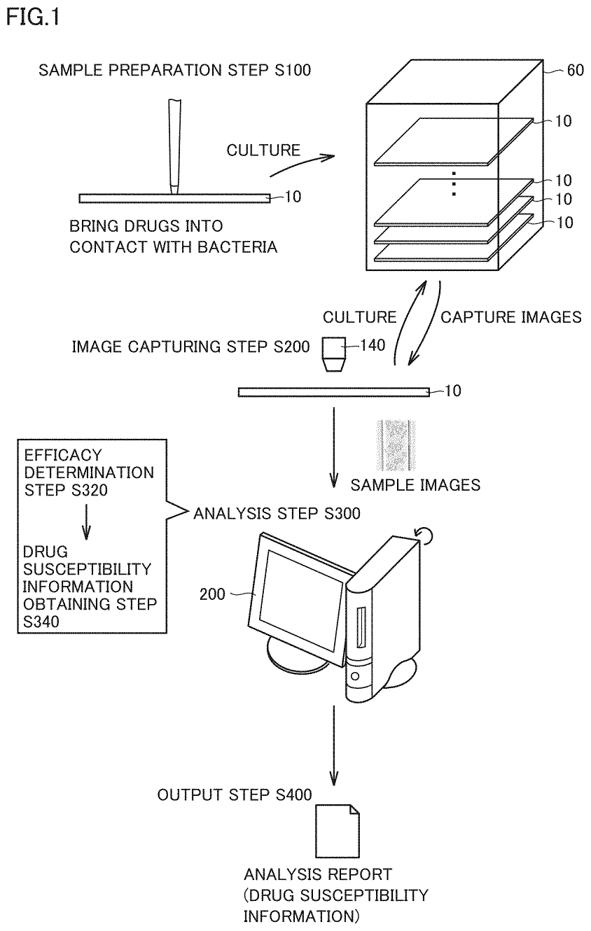

FIG. 1 shows a flow of examination according to the present embodiment. The examination according to the present embodiment includes a sample preparation step S100, an image capturing step S200, an analysis step S300, and an output step S400.

In sample preparation step S100, a test solution including bacteria is injected into a culture plate 10 on which drugs are arranged, and the drugs are brought into contact with the bacteria. Culture plate 10 into which the test solution is injected is housed in an incubator 60.

Culture plate 10 has a plurality of flow paths. Each flow path is provided with an observation point and the drugs of different types are arranged at the respective observation points. In addition, different amounts of the drugs are arranged at the respective observation points. By supplying the test solution including the bacteria to each flow path, the drugs are brought into contact with the bacteria.

In image capturing step S200, an image of each observation point on culture plate 10 is captured using a microscope camera 140, to thereby obtain image data of the samples. In addition, in image capturing step S200, culture plate 10 is housed in incubator 60 and an image of culture plate 10 is captured every prescribed time period. Culture plate 10 is housed in incubator 60 for, for example, three hours after the drugs are brought into contact with the bacteria. Then, assuming that the time of bringing the drugs into contact with the bacteria is 0 minute, culture plate 10 is taken out of incubator 60 and an image of culture plate 10 is captured at each of 0 minute, 60 minutes, 90 minutes, 120 minutes, 150 minutes, and 180 minutes.

In analysis step S300, an information processing device 200 obtains drug susceptibility information based on the images of the plurality of samples (sample images) different in conditions obtained in image capturing step S200. “Different in conditions” herein specifically means being different in at least one condition of a drug type, a drug concentration and exposure time of the bacteria. That is, in image capturing step S200, an image data set is obtained by capturing an image of each of the plurality of samples.

The drug susceptibility information is additional information about a drug that is effective against the bacteria, and is information indicating a difference in the efficacy of the drug due to being different in the at least one condition of the drug type, the drug concentration and the exposure time of the bacteria to the drug. The drug susceptibility information includes, for example, superiority or inferiority of the efficacy among the drugs, a relationship between the drug concentration and the efficacy, a relationship between the exposure time and the efficacy of the drug, and the like.

Analysis step S300 includes an efficacy determination step S320 and a drug susceptibility information obtaining step S340. In efficacy determination step S320, the efficacy of the drug against the bacteria is determined by, for example, inputting the sample images into a determination model trained by machine learning. In efficacy determination step S320, the efficacy of the drug may be determined by comparing the image data using a known image processing technique.

In drug susceptibility information obtaining step S340, the drug susceptibility information is obtained by extracting an image data subset from among the obtained image data set of the plurality of samples, the image data subset being for samples including the drug determined as being effective in efficacy determination step S320, and comparing one image with another among the image data subset in accordance with a prescribed criterion.

In output step S400, the drug susceptibility information is output as, for example, an analysis report. The analysis report may be output in the form of paper, or may be output in the form of presentation on a display.

Analysis step S300 may further include a step of determining a minimum inhibitory concentration (MIC) based on the information obtained in efficacy determination step S320.

The analysis report may include the MIC. That is, the analysis report may show the MIC and the drug susceptibility information.

[Configuration of Culture Plate]

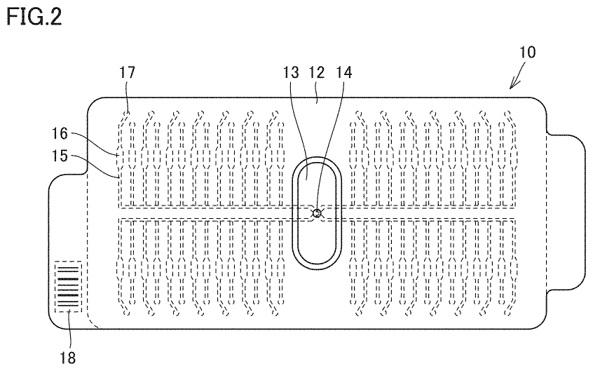

FIG. 2 is a plan view of the culture plate. Culture plate 10 includes a plate-shaped member 12 and a flow path structure. The flow path structure includes an opening portion 13, an opening 14, a micro flow path 15, an observation point 16, and an opening 17.

Opening 14 is a portion provided in opening portion 13 and allowing opening portion 13 and micro flow path 15 to communicate with each other. Namely, opening 14 is connected to one end of micro flow path 15. Using a fluid pressure, the test solution including the bacteria is injected from opening 14 into micro flow path 15. On culture plate 10 shown in FIG. 2, four micro flow paths 15 are arranged radially around opening 14.

Micro flow path 15 is configured such that the test solution can flow therethrough. Micro flow path 15 extending from opening 14 branches off to a plurality of micro flow paths 15. The test solution introduced from opening 14 flows through branched micro flow paths 15. In the present embodiment, one micro flow path 15 branches off to fourteen micro flow paths 15.

Observation point 16 is provided partway through branched micro flow path 15. Micro flow path 15 allows the test solution introduced from opening 14 to flow to observation point 16.

Observation point 16 has the drug arranged thereat, and is connected to micro flow path 15 to store the test solution introduced from micro flow path 15. At observation point 16, the test solution reacts with the drug. The drug is, for example, an antibacterial drug. The drug may be solid, or may be liquid. The drug is preliminarily placed at observation point 16. That is, the drug is placed at observation point 16 before the test solution flows into observation point 16. Observation point 16 is formed to have a rectangular parallelepiped shape. One side of observation point 16 has a length of, for example, 10 μm to 10 mm.

In FIG. 2, fifty-six (=14×4) observation points 16 are formed on plate-shaped member 12. That is, in the present embodiment, when one culture plate 10 is observed, fifty-six observation points 16 are observed using an examination device 100. Volumes of the test solution stored in fifty-six observation points 16 are the same as each other. In contrast, the types and the amounts of the drugs placed at fifty-six observation points 16 may be the same as each other, or may be different from each other. Since the volumes of the test solution stored in fifty-six observation points 16 are the same as each other, a drug concentration in each observation point 16 can be changed by placing different amounts of the drugs at observation points 16.

Plate-shaped member 12 is made of an acrylic resin such as a polymethyl methacrylate resin. A thickness of plate-shaped member 12 is not particularly limited, and is set at, for example, 1 mm to 6 mm. In addition, an identification code 18 for individually identifying culture plate 10 is assigned to plate-shaped member 12.

Identification code 18 is not limited to an optically readable code such as a one-dimensional barcode or a two-dimensional QR code (registered trademark), and may be a code that can be read by wireless communication, such as an RF tag. Identification information indicated by identification code 18 is not limited to the serial number individually assigned to culture plate 10, and may be the lot number assigned to culture plate 10.

Culture plate 10 is made mainly of an acrylic resin. Therefore, culture plate 10 has a slight individual difference due to a difference in manufacturing condition, storage condition, environmental condition during use, or the like. When a camera having a wide depth of field is used, an image that is in focus to some extent is obtained, regardless of the slight individual difference, by focusing on the same position as a position of a focal point that is focused on when capturing an image of one culture plate 10, and capturing an image of another culture plate 10. However, in the examination according to the present embodiment, microscope camera 140 having an extremely narrow depth of field is used. Therefore, an in-focus image is not obtained by focusing on the same position as a position of a focal point that is focused on when capturing an image of one culture plate 10, and capturing an image of another culture plate 10.

Accordingly, in the examination according to the present embodiment using the microscope camera having an extremely narrow depth of field, information for focusing on each observation point 16 is managed, as an image capturing condition, by the identification information indicated by individual identification code 18 assigned to each culture plate 10.

In addition, in the present embodiment, the number is assigned to each of fifty-six observation points 16. The type and the amount of the drug placed at each observation point 16 can be identified by the identification information and the number of observation point 16.

[Configuration of Examination Device Including Microscope Camera]

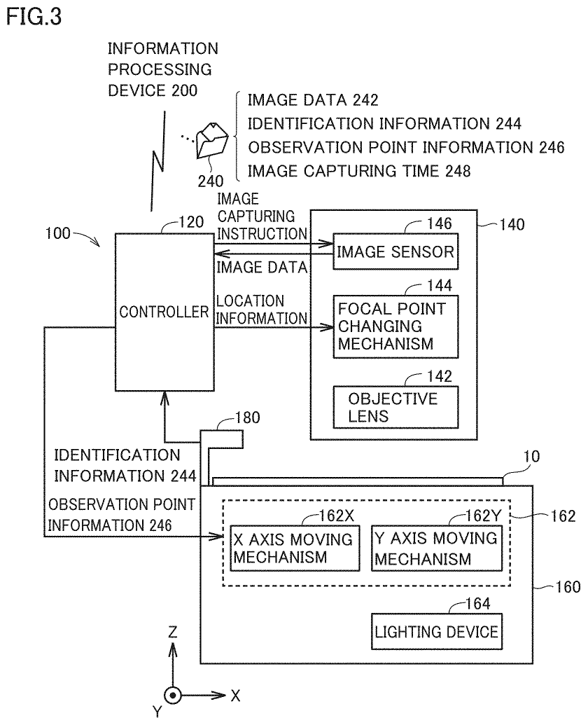

FIG. 3 shows a schematic configuration of the examination device including the microscope camera according to the present embodiment. Examination device 100 captures an image of each of the plurality of observation points provided on culture plate 10. The sample obtained by bringing the drug into contact with the test solution including the bacteria is arranged at each of the plurality of observation points.

Examination device 100 includes a controller 120, microscope camera 140, a stage 160, and a reader 180. Controller 120 is electrically connected to microscope camera 140, stage 160 and reader 180. The electrically connected devices may be partly or entirely formed of one piece.

In order to capture an image of each observation point 16 on culture plate 10, controller 120 controls each of microscope camera 140 and stage 160 based on the identification information read by reader 180.

Microscope camera 140 includes an objective lens 142, a focal point changing mechanism 144 and an image sensor 146.

Objective lens 142 magnifies a part of culture plate 10 placed on stage 160. Objective lens 142 is arbitrarily selected in accordance with an observation target.

Focal point changing mechanism 144 changes a focal point of microscope camera 140. As one example, focal point changing mechanism 144 changes the focal point of microscope camera 140 by changing a position of objective lens 142 in an optical axis direction of objective lens 142.

Image sensor 146 is a detector for capturing an image of the observation target magnified by objective lens 142, and is, for example, a charge coupled device (CCD) image sensor, a complementary metal oxide semiconductor (CMOS) image sensor or the like.

Stage 160 includes an image-capturing field-of-view changing mechanism 162 and a lighting device 164. Culture plate 10 is placed on stage 160. Lighting device 164 is transparent lighting and irradiates stage 160 with light for observation.

Image-capturing field-of-view changing mechanism 162 changes an image-capturing field of view of microscope camera 140. Image-capturing field-of-view changing mechanism 162 includes an X axis moving mechanism 162X and a Y axis moving mechanism 162Y. X axis moving mechanism 162X moves culture plate 10 placed on stage 160 in an X axis direction in FIG. 3. Y axis moving mechanism 162Y moves culture plate 10 placed on stage 160 in a Y axis direction in FIG. 3. In FIG. 3, a plane of stage 160 on which culture plate 10 is placed is defined as an X-Y plane, and an axis vertical to the X-Y plane is defined as a Z axis.

Reader 180 reads the identification information of culture plate 10. Reader 180 is, for example, a barcode reader, a QR code (registered trademark) reader or a reader adapted to a radio frequency (RF) tag, and is selected in accordance with the type of the identification code assigned to culture plate 10. Reader 180 transmits the read identification information to controller 120.

Controller 120 reads the image capturing condition corresponding to the identification information based on the identification information from reader 180, and controls microscope camera 140 and stage 160 based on the read image capturing condition, to capture an image of each observation point.

Specifically, controller 120 outputs, to image-capturing field-of-view changing mechanism 162, location information of the observation point whose image is to be captured. In accordance with the output location information, Image-capturing field-of-view changing mechanism 162 moves culture plate 10, such that the observation point whose image is to be captured is located within the image-capturing field of view of microscope camera 140.

In addition, controller 120 provides a focal point changing instruction to focal point changing mechanism 144 in accordance with the image capturing condition. At this time, controller 120 outputs, to focal point changing mechanism 144, location information when microscope camera 140 focuses on the observation point whose image is to be captured. Focal point changing mechanism 144 sets the focal point of microscope camera 140 in accordance with the output location information.

Controller 120 provides an image capturing instruction to image sensor 146 when the image-capturing field of view and the focal point of microscope camera 140 are set, and obtains image data as an observation result.

Controller 120 is communicably connected to information processing device 200. Controller 120 transmits an observation result 240 including image data 242 to information processing device 200. In addition to image data 242, observation result 240 includes identification information 244 of culture plate 10 whose image was captured, observation point information 246 indicating which observation point 16 image data 242 corresponds to, and the time (image capturing time 248) at which image data 242 was obtained.

Controller 120 and information processing device 200 may be connected to be capable of exchanging various types of data. A communication method between controller 120 and information processing device 200 may be a wireless communication method using a wireless local area network (LAN) and the like, or may be a wired communication method using a universal serial bus (USB) and the like. Controller 120 may have the function of information processing device 200.

[Hardware Configuration of Information Processing Device]

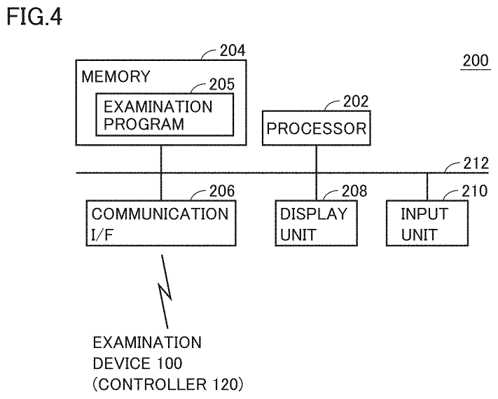

FIG. 4 is a schematic view showing one example of a hardware configuration of the information processing device. As one example, information processing device 200 is formed in accordance with a general-purpose computer architecture.

As main components, information processing device 200 includes a processor 202, a memory 204, a communication interface (I/F) 206, a display unit 208, and an input unit 210. These components are communicably connected to each other through a bus 212. Processor 202 is typically a processing unit such as a central processing unit (CPU) or a multi processing unit (MPU). Processor 202 reads and executes a program stored in memory 204, to thereby implement each process of information processing device 200 described below. In the example of FIG. 4, information processing device 200 includes a single processor. However, information processing device 200 may include a plurality of processors.

Memory 204 is implemented by a nonvolatile memory such as a random access memory (RAM), a read only memory (ROM) and a flash memory. Memory 204 stores a program executed by processor 202, data used by processor 202, or the like. For example, memory 204 stores an examination program 205 for examining the efficacy of the drugs.

Memory 204 may be a compact disc-read only memory (CD-ROM), a digital versatile disk-read only memory (DVD-ROM), a universal serial bus (USB) memory, a memory card, a flexible disk (FD), a hard disk, a solid state drive (SSD), a magnetic tape, a cassette tape, a magnetic optical disc (MO), a mini disc (MD), an integrated circuit (IC) card (excluding a memory card), an optical card, a mask ROM, or an EPROM, as long as memory 204 can record the program in a non-transitory manner in the form of being readable by information processing device 200 which is one type of computer.

Communication I/F 206 is an interface for communicating with controller 120 of examination device 100.

Display unit 208 is implemented by a liquid crystal display panel or the like. Display unit 208 displays, for example, a result of calculation made by processor 202, and the like. Input unit 210 is implemented by a mouse, a keyboard or the like. Input unit 210 receives a user operation. Information processing device 200 may include a touch panel in which display unit 208 and input unit 210 are integrated.

[Functional Configuration of Information Processing Device]

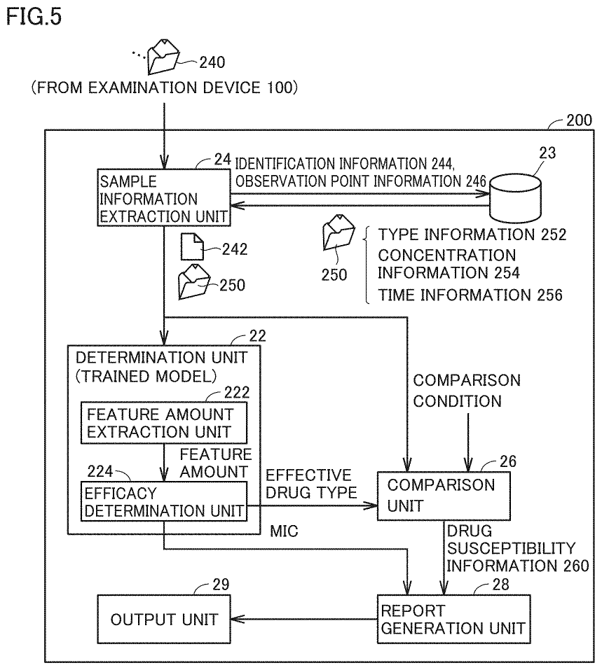

FIG. 5 is a block diagram showing one example of a functional configuration of the information processing device. Each function shown in FIG. 5 is implemented by processor 202 executing examination program 205 stored in memory 204.

Information processing device 200 includes a determination unit 22, a sample information extraction unit 24, a comparison unit 26, a report generation unit 28, and an output unit 29.

Sample information extraction unit 24 extracts sample information 250 corresponding to each observation result 240 (image data 242). Specifically, sample information extraction unit 24 obtains sample information 250 from a database 23, based on identification information 244 and observation point information 246 included in observation result 240. Sample information 250 includes type information 252, concentration information 254 and time information 256.

Type information 252 is information that can identify a drug type. Concentration information 254 is information that can identify a drug concentration. Concentration information 254 may be information indicating an amount of the drug arranged at observation point 16.

Database 23 includes the information that can identify the type and the amount of the drug arranged at each observation point 16 of each culture plate 10. Sample information extraction unit 24 obtains type information 252 and concentration information 254 from database 23, based on identification information 244 and observation point information 246.

The information that can identify the type and the amount of the drug, which is included in database 23, is generated, for example, when the drug is arranged at each observation point 16. The timing of arranging the drug may be the time of shipment of culture plate 10, or may be the time of execution of the examination after culture plate 10 is shipped. When the drug is arranged at the time of shipment, the information that can identify the type and the amount of the drug, which is included in database 23, is prestored in a server or the like that can communicate with information processing device 200. When the drug is arranged at the time of execution of the examination, the information that can identify the type and the amount of the drug is generated by the user operating input unit 210 to input the type and the amount of the drug arranged at each observation point 16.

Time information 256 indicates the time of injection of the test solution into culture plate 10 indicated by identification information 244. Sample information extraction unit 24 can obtain the exposure time of the bacteria by subtracting the time indicated by time information 256 from image capturing time 248. Time information 256 is stored in database 23 for each identification information 244.

When the test solution is injected by a machine, time information 256 is recorded by the machine for injection. When the test solution is injected by the user, time information 256 is input by the user through input unit 210.

Determination unit 22 is a model trained by machine learning (trained model). Determination unit 22 is a trained model for determining whether or not the bacteria is resistant to the drugs in the samples. Determination unit 22 includes a feature amount extraction unit 222 and an efficacy determination unit 224.

Feature amount extraction unit 222 performs preprocessing for extracting a feature amount from image data 242. For example, feature amount extraction unit 222 extracts image data 242 of a control based on sample information 250. The control is a standard sample, and is, for example, a sample made only of the test solution.

Feature amount extraction unit 222 extracts the feature amount by comparing image data 242 of the control with image data 242 of a sample other than the control. The feature amount includes, for example, a degree of extension of the bacteria with respect to the control, the number of the bacteria that increase or decrease with respect to the control, roundness of the bacteria with respect to the control, image brightness with respect to the control, image contrast with respect to the control, and the like.

Efficacy determination unit 224 determines whether or not the drug is effective, by inputting the feature amount extracted by feature amount extraction unit 222 into the trained model. For example, for each sample, efficacy determination unit 224 determines whether or not the growth of the bacteria is inhibited, based on the feature amount. When efficacy determination unit 224 determines that the growth of the bacteria is inhibited, efficacy determination unit 224 determines that the drug is effective. When efficacy determination unit 224 determines that the growth of the bacteria is not inhibited, efficacy determination unit 224 determines that the drug is not effective.

In addition, efficacy determination unit 224 determines the MIC based on the determination result. That is, determination unit 22 including efficacy determination unit 224 has the function of determining the MIC.

Efficacy determination unit 224 determines the efficacy of the drug based on the determined MIC. The efficacy of the drug is expressed, for example, by S (susceptible) and R (resistant). S indicates that the drug is effective against the bacteria. R indicates that the bacteria is resistant to the drug and the drug is not effective against the bacteria. Efficacy determination unit 224 determines the efficacy for each of the drugs of a plurality of types.

Determination unit 22 may include a neural network. In this case, determination unit 22 does not necessarily need to include feature amount extraction unit 222.

The trained model is generated based on, for example, a plurality of pieces of training data including an image indicating a state in which the bacteria is resistant to the drug and an image indicating a state in which the bacteria is not resistant to the drug.

Comparison unit 26 extracts a plurality of comparison targets from observation result 240 in accordance with a comparison condition, compares feature amounts of the extracted comparison targets, and generates drug susceptibility information 260.

Comparison unit 26 receives information indicating an effective drug type from efficacy determination unit 224 as a determination result. Comparison unit 26 identifies samples of the effective drugs, of the plurality of samples, based on the information indicating the effective drug type. In addition, comparison unit 26 extracts samples serving as comparison targets from the samples of the effective drugs, based on the comparison condition. That is, comparison unit 26 extracts the image data subset from among the obtained image data set of the plurality of samples, the image data subset being for samples including the drug determined as being effective.

Drug susceptibility information 260 is information about the efficacy of the drug and includes, for example, the concentration dependence of the drug, an antibacterial activity for each drug type, whether or not the drug has an initial antibacterial activity against the bacteria, or the like.

Report generation unit 28 generates a report for suggesting a drug suitable as an antibacterial drug, a drug administration plan and the like, based on generated drug susceptibility information 260 and the MIC included in the determination result by determination unit 22. The report includes at least an examination result list that shows the MIC and drug susceptibility information 260. The examination result list shows, for example, the presence or absence of the efficacy for each drug type. For the effective drug, the examination result list also shows the concentration dependence of the drug, the presence or absence of the initial antibacterial activity of the drug and the like together with the MIC. The examination result list may include information about a difference in antibacterial activity for each drug type.

Output unit 29 outputs the report including the examination result list generated by report generation unit 28 to, for example, display unit 208. A destination of the generated report is not limited to display unit 208. The destination may be, for example, a printer, another information processing device (processor) communicably connected to information processing device 200, a storage device such as a server communicably connected to information processing device 200, or the like.

[Comparison Condition]

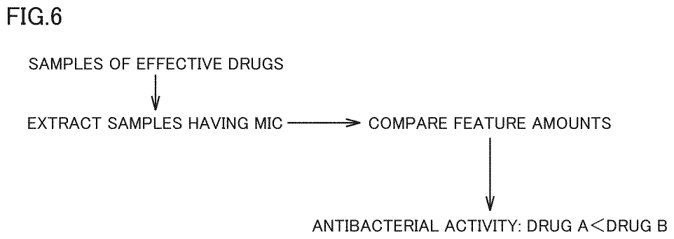

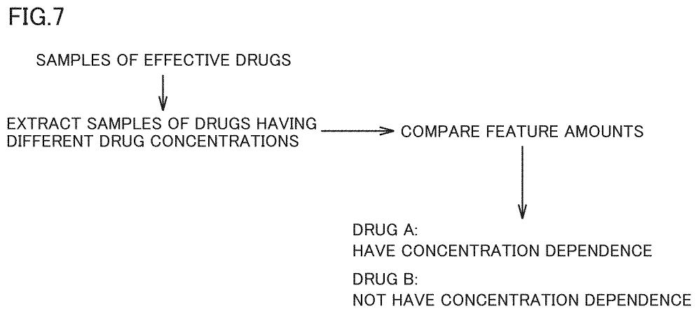

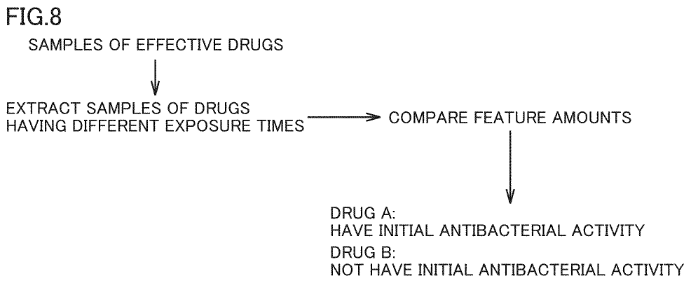

The comparison condition will be described with reference to FIGS. 6 to 8. FIG. 6 shows an example of comparison based on the drug type. FIG. 7 shows an example of comparison based on the drug concentration. FIG. 8 shows an example of comparison based on the exposure time.

(Comparison Based on Drug Type)

Referring to FIG. 6, when “samples having the MIC” are set as the extraction condition, samples having the MIC are extracted from the samples of the effective drugs. The samples having the MIC include the drug determined as being effective and are different from each other in drug type and. That is, the image data subset is extracted from among the obtained image data set of the plurality of samples including the drug determined as being effective and being different from each other in drug type. The MIC is obtained in the course of the process performed by efficacy determination unit 224. The exposure time of each of the extracted samples is preferably the same.

As to the samples having the MIC, of the identified drug types, feature amounts extracted from image data 242 of the samples are compared. The feature amount refers to, for example, the number of the bacteria that increase or decrease with respect to the control. By comparing the feature amounts, degrees of antibacterial activity of the drug types can be compared. That is, one image among the extracted image data subset is compared with another image among the extracted image data subset.

As a result, a difference in antibacterial activity between drug types is obtained as drug susceptibility information 260. For example, when a drug B has higher antibacterial activity than a drug A, it is expected that drug B is suitable as a drug to be administered.

In order to obtain the difference in antibacterial activity between drug types, determination may be made based on a plurality of types of feature amounts. Comparison unit 26 may extract a feature amount different from the feature amount obtained by the preprocessing performed by determination unit 22, to obtain the difference in antibacterial activity between drug types.

(Comparison Based on Drug Concentration)

Referring to FIG. 7, when “samples of drugs different in drug concentration” are set as the extraction condition, samples of drugs different in drug concentration are extracted from the samples of the effective drugs. For example, based on type information 252 and time information 256, samples including drug A and exposed to the bacteria for the specific exposure time are extracted from the samples of the effective drugs. As a result, the samples of drug A having the specific exposure time and having the different concentrations are extracted. That is, the image data subset is extracted from among the obtained image data set of the plurality of samples including the drug determined as being effective and being different from each other in drug concentration.

Feature amounts extracted from image data 242 of the extracted samples are compared. The feature amount refers to, for example, the number of the bacteria that increase or decrease with respect to the control. By comparing the feature amounts, the concentration dependence of the antibacterial activity is known. For example, when an amount of decrease in the number of the bacteria becomes larger as the concentration becomes higher, it is determined that the drug has the concentration dependence. In contrast, when the amount of decrease in the number of the bacteria does not change even if the concentration becomes higher, it is determined that the drug does not have the concentration dependence. Comparison unit 26 determines the concentration dependence for each drug. That is, comparison unit 26 compares one image with another among the extracted image data subset.

For example, when it is determined that drug A has the concentration dependence, it can be seen that the antibacterial activity becomes higher by increasing an amount of drug A to be administered. In contrast, when it is determined that drug B does not have the concentration dependence, it can be seen that the antibacterial activity does not change even if an amount of drug B to be administered is increased.

In order to determine the concentration dependence of the antibacterial activity, determination may be made based on a plurality of types of feature amounts. Comparison unit 26 may extract a feature amount different from the feature amount obtained by the preprocessing performed by determination unit 22, to determine the concentration dependence of the antibacterial activity.

(Comparison Based on Exposure Time)

Referring to FIG. 8, when “samples of drugs different in exposure time” are set as the extraction condition, samples of drugs different in exposure time are extracted from the samples of the effective drugs. That is, the image data subset is extracted from among the obtained image data set of the plurality of samples including the drug determined as being effective and being different from each other in exposure time. For example, based on type information 252 and concentration information 254, samples including drug A, having different exposure times, and having a common drug concentration are extracted from the samples of the effective drugs. As a result, the samples of drug A having the different exposure times and having the specific concentration are extracted.

Feature amounts extracted from image data 242 of the extracted samples are compared. The feature amount refers to, for example, the number of the bacteria that increase or decrease with respect to the control. By comparing the feature amounts, it is known whether or not the antibacterial activity appears from the beginning of exposure. Specifically, when the bacteria greatly decrease with respect to the control from the beginning of exposure, it is estimated that the antibacterial activity appears from the beginning of exposure, and thus, it is determined that the drug has the initial antibacterial activity. Comparison unit 26 determines, for each drug, whether or not the drug has the initial antibacterial activity. That is, comparison unit 26 compares one image with another among the extracted image data subset.

For example, when it is determined that drug A has the initial antibacterial activity, it can be seen that the effect of administering drug A can be recognized in the beginning of administration. In contrast, when it is determined that drug B has the initial antibacterial activity, it can be seen that the effect of administering drug B cannot be recognized in the beginning of administration.

In order to determine whether or not the drug has the initial antibacterial activity, determination may be made based on a plurality of types of feature amounts. Comparison unit 26 may extract a feature amount different from the feature amount obtained by the preprocessing performed by determination unit 22, to determine whether or not the drug has the initial antibacterial activity.

[Aspects]

It is understood by a person skilled in the art that the above-described embodiment and modifications thereof are provided as specific examples of the following aspects.

(Clause 1)

An examination method according to one aspect is an examination method for examining efficacy of a drug against bacteria. The examination method comprising: obtaining a plurality of samples, each of the plurality of samples being obtained by bringing a drug into contact with the bacteria; obtaining an image data set by capturing an image of each of the plurality of samples, the plurality of samples being different from each other in at least one condition of a drug type, a drug concentration and exposure time of the bacteria to the drug; determining the efficacy of the drug against the bacteria based on the obtained image data set; and obtaining information indicating a difference in the efficacy of the drug due to being different in the at least one condition of the drug type, the drug concentration and the exposure time of the bacteria to the drug, by extracting an image data subset from among the obtained image data set of the plurality of samples, the image data subset being for samples including the drug determined as being effective, and comparing one image with another among the image data subset in accordance with a prescribed criterion.

With such a configuration, the information indicating the difference in the efficacy of the drug is obtained for the drug determined as being effective, by comparing the image data of the plurality of samples including the drug determined as being effective, in accordance with the prescribed criterion.

(Clause 2)

The examination method according to clause 1 further comprises determining a minimum inhibitory concentration of the drug against the bacteria based on a result obtained by determining the efficacy of the drug against the bacteria.

With such a configuration, the effective drug can be identified based on the minimum inhibitory concentration.

(Clause 3)

The examination method according to clause 2 further comprises presenting the obtained information indicating the difference in the efficacy, together with the minimum inhibitory concentration.

With such a configuration, the two information is presented together, and thus, the user can identify the effective drug and check the difference in the efficacy about the identified drug at the same time.

(Clause 4)

In the examination method according to any one of clauses 1 to 3, in the obtaining information indicating a difference in the efficacy, a degree of the efficacy based on the drug type is obtained by extracting the image data subset from among the obtained image data set of the plurality of samples, the image data subset being for samples different from each other in the drug type, and comparing one image with another among the image data subset.

With such a configuration, the degree of the efficacy based on the drug type is obtained, and thus, selection of the drug type to be administered can be assisted.

(Clause 5)

In the examination method according to any one of clauses 1 to 4, in the obtaining information indicating a difference in the efficacy, a relationship between a difference in the drug concentration and a degree of the efficacy is obtained by extracting the image data subset from among the obtained image data set of the plurality of samples, the image data subset being for samples different from each other in the drug concentration, and comparing one image with another among the image data subset.

With such a configuration, the relationship between the difference in the drug concentration and the degree of the efficacy is obtained, and thus, selection of the concentration of the drug to be administered can be assisted.

(Clause 6)

In the examination method according to any one of clauses 1 to 5, in the obtaining information indicating a difference in the efficacy, initial susceptibility of the drug is obtained by extracting the image data subset from among the obtained image data set of the plurality of samples, the image data subset being for samples different from each other in the exposure time of the bacteria, and comparing one image with another among the image data subset.

With such a configuration, the initial susceptibility of the drug is obtained, and thus, an indicator of the timing of determining a drug administration result can be provided.

(Clause 7)

In the examination method according to any one of clauses 1 to 6, in the determining the efficacy of the drug, the efficacy of the drug against the bacteria is determined by inputting the image data of the plurality of samples different in the condition into a determination model trained by machine learning.

(Clause 8 )

In the examination method according to clause 7, the determining the efficacy of the drug includes extracting a feature amount about the bacteria included in the samples.

(Clause 9)

In the examination method according to clause 7, the determination model includes a convolutional neural network.

(Clause 10 )

In the examination method according to any one of clauses 1 to 9, the plurality of samples are obtained by supplying a test solution including the bacteria to each of a plurality of flow paths formed in a device, the drug being arranged in each of the plurality of flow paths.

With such a configuration, the plurality of samples can be obtained simply by supplying the test solution to the flow paths, and thus, the samples can be easily prepared.

(Clause 11)

A program according to one aspect is a program for examining efficacy of a drug against bacteria based on an image data set, the image data set being obtained by capturing an image of each of a plurality of samples, each of the plurality of samples being obtained by bringing a drug into contact with the bacteria, the plurality of samples being different from each other in at least one condition of a drug type, a drug concentration and exposure time of the bacteria to the drug. The program causes the processor to perform the functions of: determining a minimum inhibitory concentration of the drug against the bacteria; obtaining information indicating a difference in the efficacy of the drug due to being different in the at least one condition of the drug type, the drug concentration and the exposure time of the bacteria to the drug; and outputting an examination result list that shows the minimum inhibitory concentration and the information indicating the difference in the efficacy.

With such a configuration, additional information, i.e., the information indicating the difference in the efficacy of the drug due to being different in the at least one condition of the drug type, the drug concentration and the exposure time of the bacteria to the drug, is obtained. Furthermore, the examination result list that shows the information indicating the difference in the efficacy of the drug and the minimum inhibitory concentration is output, and thus, the user can identify the effective drug and check the difference in the efficacy about the identified drug at the same time.

(Clause 12)

A destination of the examination result list includes at least one of a printer, a display device, a processor different from the processor that performs the above-described program, and a storage device communicably connected to the processor that performs the above-described program.

(Clause 13)

An examination system according to one aspect includes the processor that performs the program as recited in clause 11 or 12.

(Clause 14)

The examination system according to clause 13 may further comprise an image capturing device that captures an image of each of the plurality of samples, the plurality of samples being different from each other in the at least one condition of the drug type, the drug concentration and the exposure time of the bacteria to the drug, to thereby obtain image data. In this case, the function of determining a minimum inhibitory concentration includes the function of determining the minimum inhibitory concentration based on the image data.

(Clause 15)

In the examination system according to clause 13 or 14, the function of obtaining information indicating a difference in the efficacy includes the function of obtaining the information indicating the difference in the efficacy, by extracting an image data subset from among the obtained image data set of the plurality of samples, the image data subset being for samples including the drug determined as being effective, and comparing one image with another among the image data subset in accordance with a prescribed criterion.

(Clause 16)

A computer readable medium according to one aspect has the program as recited in clause 11 or 12 stored therein in a non-transitory manner.

While the embodiment of the present disclosure has been described, it should be understood that the embodiment disclosed herein is illustrative and non-restrictive in every respect. The scope of the present disclosure is defined by the terms of the claims and is intended to include any modifications within the scope and meaning equivalent to the terms of the claims.

What is claimed is:

1. An examination method for examining efficacy of a drug against bacteria, the examination method comprising:
obtaining a plurality of samples, each of the plurality of samples being obtained by bringing a drug into contact with the bacteria;
obtaining an image data set by capturing an image of each of the plurality of samples, the plurality of samples being different from each other in at least one condition of a drug type, a drug concentration and exposure time of the bacteria to the drug;
determining the efficacy of the drug against the bacteria based on the obtained image data set; and
obtaining information indicating a difference in the efficacy of the drug due to being different in the at least one condition of the drug type, the drug concentration and the exposure time of the bacteria to the drug, by
extracting an image data subset from among the obtained image data set of the plurality of samples, the image data subset being for samples including the drug determined as being effective, and
comparing one image with another among the image data subset in accordance with a prescribed criterion.

2. The examination method according to claim 1, further comprising
determining a minimum inhibitory concentration of the drug against the bacteria based on a result obtained by determining the efficacy of the drug against the bacteria.

3. The examination method according to claim 2, further comprising
presenting the obtained information indicating the difference in the efficacy, together with the minimum inhibitory concentration.

4. The examination method according to claim 1, wherein
in the obtaining information indicating a difference in the efficacy, a degree of the efficacy based on the drug type is obtained by
extracting the image data subset from among the obtained image data set of the plurality of samples, the image data subset being for samples different from each other in the drug type, and
comparing one image with another among the image data subset.

5. The examination method according to claim 1, wherein
in the obtaining information indicating a difference in the efficacy, a relationship between a difference in the drug concentration and a degree of the efficacy is obtained by
extracting the image data subset from among the obtained image data set of the plurality of samples, the image data subset being for samples different from each other in the drug concentration, and
comparing one image with another among the image data subset.

6. The examination method according to claim 1, wherein
in the obtaining information indicating a difference in the efficacy, initial susceptibility of the drug is obtained by
extracting the image data subset from among the obtained image data set of the plurality of samples, the image data subset being for samples different from each other in the exposure time of the bacteria, and
comparing one image with another among the image data subset.

7. The examination method according to claim 1, wherein
in the determining the efficacy of the drug, the efficacy of the drug against the bacteria is determined by inputting the image data of the plurality of samples different in the condition into a determination model trained by machine learning.

8. The examination method according to claim 7, wherein
the determining the efficacy of the drug includes extracting a feature amount about the bacteria included in the samples.

9. The examination method according to claim 7, wherein
the determination model includes a convolutional neural network.

10. The examination method according to claim 1, wherein
the plurality of samples are obtained by supplying a test solution including the bacteria to each of a plurality of flow paths formed in a device, the drug being arranged in each of the plurality of flow paths.

11. An examination system including a processor having a program for examining efficacy of a drug against bacteria based on an image data set installed thereon, the image data set being obtained by capturing an image of each of a plurality of samples, each of the plurality of samples being obtained by bringing a drug into contact with the bacteria, the plurality of samples being different from each other in at least one condition of a drug type, a drug concentration and exposure time of the bacteria to the drug, the program causing the processor to perform the functions of:
determining a minimum inhibitory concentration of the drug against the bacteria;
obtaining information indicating a difference in the efficacy of the drug due to being different in the at least one condition of the drug type, the drug concentration and the exposure time of the bacteria to the drug; and
outputting an examination result list that shows the minimum inhibitory concentration and the information indicating the difference in the efficacy.

12. The examination system according to claim 11, wherein
a destination of the examination result list includes at least one of a printer, a display device, a processor different from the processor, and a storage device communicably connected to the processor.

13. The examination system according to claim 12, further comprising
an image capturing device that captures an image of each of the plurality of samples, the plurality of samples being different from each other in the at least one condition of the drug type, the drug concentration and the exposure time of the bacteria to the drug, to thereby obtain image data, wherein
the function of determining a minimum inhibitory concentration includes the function of determining the minimum inhibitory concentration based on the image data.

14. The examination system according to claim 12, wherein
the function of obtaining information indicating a difference in the efficacy includes the function of obtaining the information indicating the difference in the efficacy, by
extracting an image data subset from among the obtained image data set of the plurality of samples, the image data subset being for samples including the drug determined as being effective, and
comparing one image with another among the image data subset in accordance with a prescribed criterion.

15. A non-transitory computer readable recording medium having a program for examining efficacy of a drug against bacteria based on an image data set recorded thereon, the image data set being obtained by capturing an image of each of a plurality of samples, each of the plurality of samples being obtained by bringing a drug into contact with the bacteria, the plurality of samples being different from each other in at least one condition of a drug type, a drug concentration and exposure time of the bacteria to the drug, the program causing a processor to perform the functions of:
determining a minimum inhibitory concentration of the drug against the bacteria;
obtaining information indicating a difference in the efficacy of the drug due to being different in the at least one condition of the drug type, the drug concentration and the exposure time of the bacteria to the drug; and
outputting an examination result list that shows the minimum inhibitory concentration and the information indicating the difference in the efficacy.

16. The non-transitory computer readable recording medium according to claim 15, wherein
a destination of the examination result list includes at least one of a printer, a display device, a processor different from the processor, and a storage device communicably connected to the processor.
